(19) United States
(12) Patent Application Publication
(10) Pub. No.: US 2022/0389520 A1
(43) Pub. Date: Dec. 8, 2022

(54) SYSTEMS AND METHODS FOR INTEGRATED ANALYSIS OF BLOOD AND SURGICAL DRAIN FLUID BIOMARKERS

(71) Applicant: Washington University, St. Louis, MO (US)

(72) Inventors: Jose Zevallos, St. Louis, MO (US); Aadel Chaudhuri, St. Louis, MO (US)

(73) Assignee: Washington University, St. Louis, MO (US)

(21) Appl. No.: 17/834,663

(22) Filed: Jun. 7, 2022

Related U.S. Application Data
(60) Provisional application No. 63/197,719, filed on Jun. 7, 2021.

(51) Int. Cl. C12Q 1/6886; A61K 45/06

(52) U.S. Cl. CPC C12Q 1/6886; A61K 45/06; C12Q 1/6806

(57) ABSTRACT

A method for detecting at least one systemic condition and at least one locoproximal condition within a surgical site of a subject following a surgery is disclosed. The method includes obtaining a surgical drainage fluid sample from the surgical site and a blood sample from the subject, isolating analyte-containing portions from the surgical drainage fluid sample and the blood sample, and detecting and quantifying at least a portion of the analytes within the analyte-containing portions to produce at least one assay result, wherein the at least one assay result is indicative of the at least one systemic condition and the at least one locoproximal condition within a surgical site of a subject. At least one assay result may be provided to a practitioner and used to select at least one additional treatment.

### CROSS-REFERENCE TO RELATED APPLICATIONS

This application claims priority from U.S. Provisional Application Ser. No. 63/197,719 filed on Jun. 7, 2021, which is incorporated herein by reference in its entirety.

### STATEMENT REGARDING FEDERALLY SPONSORED RESEARCH OR DEVELOPMENT

This invention was made with government support under VA ID2021-532 awarded by the U.S. Department of Veterans Administration (VA). The government has certain rights in the invention.

### MATERIAL INCORPORATED-BY-REFERENCE

Not applicable.

### FIELD OF THE DISCLOSURE

The present disclosure generally relates to systems and methods of monitoring a post-surgical patient to assess surgical outcomes and potential complications, as well as to guide the selection of post-surgical follow-up treatments.

### BACKGROUND OF THE DISCLOSURE

For patients undergoing surgical treatments, such as resectioning surgery, dissection surgery, excision surgery, and transplantation surgery, it may be challenging to monitor the patient to assess the surgical outcome, to detect the development of complications associated with the surgery, and/or to select additional treatments. Depending on the extent of the surgery and condition of the patient, it may be inadvisable to subject the patient to any of the various medical imaging modalities, and these modalities may not provide sufficient information to assess at least some of the concerns of a medical practitioner regarding the post-surgical condition of the patient. Biomarkers isolated from blood samples of the patient may provide some information regarding the post-surgical patient's condition, but typically any biomarkers released from a surgical site are diluted by the blood volume and transported by blood flow within the circulatory vessels to regions distal to the surgical site. As a consequence, blood biomarkers may be present at very low concentrations and may require a highly sensitive assay, if indeed the concentrations are above an assay's lowest detectable concentration. In addition, biomarkers detected in a patient blood sample may originate from sources other than the surgical site. Consequently, blood-based biomarkers are better suited for the evaluation of a patient's systemic state, rather than conditions at a surgical site.

Surgical drain fluid represents a potentially rich source of biomarkers indicative of a post-surgical patient's condition and prognosis. Surgical drain fluid originates at the surgery site and is drained from the patient without further dilution. Surgical drain fluid biomarkers are typically present at higher concentrations than corresponding blood concentrations. Further, biomarkers in the surgical drain fluid may have a higher range of concentrations and therefore may be detectable using assays with higher minimum detection limits.

### SUMMARY OF THE DISCLOSURE

In one aspect, a method for detecting at least one systemic condition and at least one locoproximal condition within a surgical site of a subject following a surgery is disclosed. The method includes obtaining a surgical drainage sample from the surgical site and a blood sample from the subject, isolating analyte-containing portions from the surgical drainage sample and the blood sample, detecting and quantifying at least a portion of analytes within the analyte-containing portions to produce at least one assay result, and providing the at least one assay result to a practitioner. The least one assay result is indicative of the at least one systemic condition and the at least one locoproximal condition within a surgical site of a subject. In some aspects, the surgery is selected from a resectioning surgery, a dissection surgery, an excision surgery, a transplant surgery, a reconstructive surgery, and any combination thereof. In some aspects, the analyte-containing portions isolated from the surgical drainage sample and the blood sample each comprise cfDNA, RNA, exosomes, tumor cells, immune cells, bacterial nucleic acids, viral nucleic acids, proteins, and any combination thereof. In some aspects, the analytes within the analyte-containing portions are detected and quantified using at least one assay selected from whole genome sequencing, next generation DNA sequencing, next generation RNA sequencing, PCR, Western blot targeted capture, multiplex PCR, methylation & 16S droplet PCR, and any combination thereof. In some aspects, isolating the analyte-containing portions from the surgical drainage sample and the blood sample comprises filtering the samples, centrifuging the samples, contacting the sample with chromatography media, and any combination thereof. In some aspects, the method further includes selecting an additional treatment based on the at least one assay result. In some aspects, the additional treatment is selected from radiotherapy, chemotherapy, follow-up surgery, active surveillance with imaging, antibiotic therapy, antiviral therapy, and any combination thereof. In some aspects, obtaining the sample from the subject further includes capturing a surgical drainage from a drainage tube associated with the surgery. In some aspects, obtaining the sample from the subject further comprises capturing a surgical drainage from the drainage tube within about 24 hours of the surgery.

In another aspect, a method of selecting at least one additional treatment for a subject following a surgery is disclosed. The method includes obtaining a surgical drainage sample from the surgical site and a blood sample from the subject, isolating analyte-containing portions from the surgical drainage sample and the blood sample, detecting and quantifying at least a portion of analytes within the analyte-containing portions to produce at least one assay result, and selecting the at least one additional treatment based on the at least one systemic condition and at least one locoproximal condition detected in the subject. The least one assay result is indicative of the at least one systemic condition and the at least one locoproximal condition within a surgical site of a subject. In some aspects, the at least one additional treatment is selected from radiotherapy, chemotherapy, follow-up surgery, active surveillance with imaging, antibiotic therapy, antiviral therapy, and any combination thereof. In some aspects, the surgery is selected from a resectioning surgery, a dissection surgery, an excision surgery, a transplant surgery, a reconstructive surgery, and any combination thereof. In some aspects, the analyte-containing portions isolated from the surgical drainage sample and the blood sample each comprise cfDNA, RNA, exosomes, tumor cells, immune cells, bacterial nucleic acids, viral nucleic acids, proteins, and any combination thereof. In some aspects, the analytes within the analyte-containing portions are detected and quantified using at least one assay selected from whole genome sequencing, next generation DNA sequencing, next generation RNA sequencing, PCR, Western blot targeted capture, multiplex PCR, methylation & 16S droplet PCR, and any combination thereof. In some aspects, isolating the analyte-containing portions from the surgical drainage sample and the blood sample comprises filtering the samples, centrifuging the samples, contacting the sample with chromatography media, and any combination thereof. In some aspects, obtaining the sample from the subject further includes capturing a surgical drainage from a drainage tube associated with the surgery. In some aspects, obtaining the sample from the subject further comprises capturing a surgical drainage from the drainage tube within about 24 hours of the surgery.

In an additional aspect, a method for selecting at least one adjuvant treatment for oropharyngeal squamous cell carcinoma (OPSCC) following a resection surgery is disclosed. The method includes obtaining a surgical drainage fluid sample from the surgical site and a blood sample from the subject, isolating analyte-containing portions from the surgical drainage fluid sample and the blood sample, detecting and quantifying HPV viral DNA levels within the analyte-containing portions to produce at least one assay result, and selecting the at least one adjuvant treatment based on the at least one at least one assay result. The at least one assay result is indicative of oropharyngeal squamous cell carcinoma (OPSCC) within the subject. In some aspects, selecting the at least one adjuvant treatment based on the at least one at least one assay result further includes selecting active surveillance with imaging or no adjuvant treatment if both plasma and SDF HPV levels are below a threshold value, or selecting an adjuvant treatment comprising at least one of radiotherapy, chemotherapy, follow-up surgery, and any combination thereof if at least one of the plasma HPV level and the SDF HPV level are above a threshold value. In some aspects, the method further includes predicting a time of recurrence or a survival period of the patient based on a predetermined correlation between at least one of the plasma HPV level, the SDF HPV level, and any combination thereof.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a flow chart illustrating the steps of a method for detecting at least one surgery-related condition within a surgical site of a subject following a surgery in accordance with one aspect of the disclosure.

FIG. 2 is a schematic diagram illustrating the isolation and preservation of a nucleic acid-containing portion of a sample.

FIG. 3 is a schematic of the plasmid used in the method disclosed herein.

FIG. 4A is a graph of a standard curve for an assay used in the method disclosed herein.

FIG. 4B is a graph of the concentration vs. fraction positive samples in the experiments to determine the limit of detection of the assay used in the method disclosed herein.

FIG. 5 contains graphs illustrating the efficiency of qPCR technology used in the method disclosed herein.

FIG. 6 is a graph showing several representative graphs of fluorescence vs. cycle number from a PCR experiment.

FIG. 7A is a graph of the percent of the number of wells within which HPV was detected for several different HPV copy numbers using a Thermo master mix reagent.

FIG. 7B is a graph of the percent of the number of wells within which HPV was detected for several different HPV copy numbers using an IDT master mix reagent.

FIG. 8 is a graph of HPV copies in DNA from drainage fluid (DF) samples harvested at different times.

FIG. 9A is a graph of HPV copies detected in plasma and surgical drainage fluid (SDF) samples.

FIG. 9B is a graph of the percent of plasma and surgical drainage fluid samples within which HPV was detected, broken down by samples that are within and beneath the level of detection.

FIG. 10A is a graph summarizing the percent of samples in which HPV was detected in N0 and N1/2 graded lymph nodes broken down by samples that are within and beneath the level of detection.

FIG. 10B is a graph of the number of HPV copies in drainage fluid from N0 and N1/2 graded lymph nodes.

FIG. 11A is a ROC graph of the sensitivity vs. the specificity for DNA assays from drainage fluid.

FIG. 11B is a graph differentiating samples with high and low amounts of HPV found in drainage fluid from N0 and N1/2 lymph nodes.

FIG. 12 is a graph of HPV levels in postoperative surgical drain fluid (SDF) versus surgical pathology classifications. All 84 SDF samples were categorized according to the local pathological diagnosis of the neck from which they were extracted; ENE+N1 or N2, ENE−N1 or N2, and N0. ENE+N1/N2 had the significantly highest HPV levels in SDF (median 145 copies/uL), followed by ENE−N1/N2 (median 7 copies/uL), and lastly, the N0 necks (median ND). ND=not detected.

FIG. 13A is a ROC graph of sensitivity versus specificity for DNA assays from drainage fluid.

FIG. 13B is a graph differentiating samples with high and low amounts of HPV found in drainage fluid from N1/2 ENE negative and N1/2 ENE positive lymph nodes.

FIG. 14A is a graph of the number of HPV copies detected in variously graded lymph nodes.

FIG. 14B is a graph of the percentage of samples wherein HPV was detected in variously graded lymph nodes.

FIG. 15 is a graph of HPV levels in postoperative surgical drain fluid (SDF) grouped by adjuvant treatment type. SDF was collected postoperatively (the SDF with higher HPV copies was collected from patients with bilateral neck dissections). There was a non-significant elevation in SDF HPV levels from patients who went on to receive chemoradiation therapy (CRT) compared to only radiation therapy (XRT) (median 263 vs. 18 copies/uL; p=0.56). SDF from patients in the CRT group was significantly enriched for HPV compared to no treatment (No Tx) (median 263 copies/uL vs. ND; p=0.026). The single patient that recurred in the No Tx group was deemed to be high-risk but declined adjuvant radiotherapy. ND=not detected.

FIG. 16A is a graph of the number of HPV copies found in tumors, drainage fluid, and plasma.

FIG. 16B is another graph of the number of HPV copies found in tumors, drainage fluid, and plasma.

FIG. 17A is a graph of the number of HPV copies found in tumor drainage fluid, and plasma in patients with double-positive surgical drainage fluid.

FIG. 17B is a graph of the number of HPV copies found in tumor, drainage fluid, and plasma in high-risk path patients with double-positive surgical drainage fluid.

FIG. 18A is a graph of the number of HPV copies found in tumor, drainage fluid, and plasma in low-risk path patients with double-positive surgical drainage fluid.

FIG. 18B is a graph of the number of HPV copies found in tumor, drainage fluid, and plasma in low-risk path patients with double-positive surgical drainage fluid.

FIG. 19A is a graph of the number of HPV copies found in tumor, drainage fluid, and plasma in high-risk path patients with double-negative surgical drainage fluid.

FIG. 19B is a graph of the number of HPV copies found in tumor, drainage fluid, and plasma in high-risk path patients with double-positive surgical drainage fluid.

FIG. 20A is a graph comparing the number of HPV copies over time in high and low-risk patients.

FIG. 20B is a survival curve for patients with HPV positive and negative surgical drainage fluid.

There are shown in the drawings arrangements, which are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

Tumor-associated nucleic acids in surgical drain fluid have been demonstrated to be markers of minimal residual disease after surgery. In various aspects, a method of measuring locoregional and distant residual disease after surgery by the analysis of biomarkers in surgical drain fluid as well as blood-based biomarkers is disclosed. In some aspects, tumor-associated nucleic acids are detected in a patient's plasma and surgical drain fluid (SDF) to assess for the systemic risk of metastasis and minimal residual disease within the surgical region, respectively. In some aspects, the disclosed method provides for molecular restaging after cancer surgery. In other aspects, the method provides for the selection of a post-surgical adjuvant treatment based on the detected levels of plasma and surgical drain fluid (SDF) biomarkers after completion of the surgery. The added value of combining surgical drain fluid and blood-based markers to assess the condition of the patient both within the region of surgery and systemically is described in additional detail in the Examples provided herein.

In various aspects, systems and methods for monitoring at least one condition of a post-surgical patient based on a combined analysis of surgical drain fluid and blood samples obtained from the patient following a surgical procedure. Without being limited to any particular theory, biomarkers within the surgical drain fluid are thought to be indicative of local conditions within the surgical region, and biomarkers within the blood are thought to be indicative of a patient's systemic condition. The disclosed systems and methods provide an objective measure of locoregional minimal residual disease after surgery. The disclosed systems and methods further provide for molecular restaging in the postoperative period that separately encompasses both locoregional and distant diseases. Molecular restaging in this manner is particularly useful for use in adjuvant therapy decision-making after surgery as described herein.

In various aspects, the disclosed systems and methods detect and quantify blood-based and surgical drain fluid (SDF) biomarkers that are indicative of a variety of different aspects of the systemic and local wound environments following a surgical procedure. In these aspects, the systems and methods disclosed herein are suitable for use in conjunction with a variety of different surgical procedures including, but not limited to, resectioning surgery, dissection surgery, excision surgery, transplant surgery, reconstructive surgery, and any other suitable surgery type without limitation.

In some aspects, the disclosed systems and methods may be used to guide a practitioner in the selection of adjuvant therapy following surgery for a variety of disorders including, but not limited to, cancer. In other aspects, the disclosed systems and methods may be used for “molecular restaging” following a surgical procedure for the treatment of cancer. “Molecular restaging”, as used herein, refers to a method of analyzing and comparing blood-based and SDF-based biomarker levels to assess minimal residual cancer within the surgical region as well as the risk of metastasis. In various aspects, molecular restaging is based on biomarkers rather than other diagnostic methods such as medical imaging. In some aspects, molecular restaging may replace or supplement medical imaging and other diagnostic information used by a practitioner to perform post-surgical restaging of cancer, determine a patient's prognosis, and/or select one or more adjuvant treatments following surgery.

In various aspects, a patient's systemic and locoregional conditions may be determined based on the concentrations of blood-based biomarkers from a patient's blood sample and SDF-based biomarkers from a patient's SDF sample, respectively, as measured using the disclosed systems and methods. In some aspects, each blood-based and SDF-based biomarker may be compared to its corresponding threshold value or range of values to classify a patient's condition. In other aspects, a patient's condition may be determined using a correlation of a blood-based or SDF-based biomarker with a condition. By way of non-limiting example, an SDF-based cancer biomarker may be used to determine the risk of cancer recurrence based on a correlation of SDF biomarker concentration with the risk of cancer recurrence within a population of patients receiving cancer surgery.

In other aspects, at least a portion of the blood-based and SDF-based biomarker concentrations may be mathematically combined to produce a summary value or index used to classify a patient's condition. Non-limiting examples of mathematical combinations of biomarker concentrations used to produce a summary value or index include sums, differences, products, ratios, correlation coefficients, and any other suitable mathematical combination or function. In some aspects, one or more biomarker concentrations may be independently scaled to modulate the contributions of individual biomarker concentrations to the summary value or index. In some aspects, a summary value or index may be used to determine a patient's condition based on a threshold value, range of values, or a correlation of the summary value or index with a patient's condition.

In various aspects, the threshold values or value ranges may be determined empirically based on the analysis of a plurality of blood and SDF samples from a population of subjects with known conditions.

In various aspects, the disclosed systems and methods may be further used to select a post-surgical adjuvant treatment based on the patient's condition. Without being limited to any particular theory, the classification of local and systemic conditions within the post-surgical patient based on SDF and blood biomarkers, respectively, provides a robust evaluation of the patient to replace or supplement conventional diagnostic information such as medical images.

By way of non-limiting example, SDF and blood biomarkers may be used to determine a cancer patient's risk of recurrence/minimal residual disease and risk of metastasis, respectively. In these examples, these risks may influence a practitioner's selection of post-surgical adjuvant treatments ranging from relatively mild treatments for low-risk patients to aggressive treatment if high risks of cancer recurrence and/or metastasis are indicated. In this non-limiting example, the detection of low biomarker levels in both the SDF and plasma of a post-surgical patient obtained using the disclosed method is indicative of minimal residual cancer within the surgical region as well as low risk of metastasis, and the patient may be classified as low-risk status, and adjuvant treatment appropriate for this low-risk level may be selected. The detection of high biomarker levels in the SDF and low biomarker levels in the plasma of a post-surgical patient obtained using the disclosed method is indicative of a high risk of recurrence of cancer within the surgical region as well as a low risk of metastasis, and the patient may be classified and treated accordingly. The detection of low biomarker levels in the SDF and high biomarker levels in the plasma of a post-surgical patient obtained using the disclosed method is indicative of a low risk of recurrence of cancer within the surgical region as well as a high risk of metastasis, and the patient may be classified and treated accordingly. By way of another additional non-limiting example, the detection of high biomarker levels in the SDF and plasma of a post-surgical patient obtained using the disclosed method is indicative of a high risk of recurrence of cancer within the surgical region as well as a high risk of metastasis, and the patient may be classified and treated accordingly.

In various aspects, at least one blood biomarker and at least one SDF biomarker are measured and analyzed using the disclosed systems and methods. In some aspects, matched blood and SDF biomarkers may be measured and analyzed. By way of non-limiting example, the same tumor-associated genetic material may be detected and quantified in both the blood and SDF samples to assess the risks of metastasis and recurrence of cancer, respectively. In other aspects, at least a portion of the blood biomarkers may be different from the SDF biomarkers measured and analyzed using the disclosed systems and methods. By way of non-limiting example, bacterial genetic material may be detected in the SDF sample to determine the risk of an infection within the surgical region, but not in the blood sample.

In various aspects, the blood and SDF biomarkers may be any suitable biomarker indicative of any aspect of the patient's condition without limitation. Non-limiting examples of suitable types of blood and SDF biomarkers used in the disclosed systems and methods include tumor cells, immune cells, bacterial cells, viral host cells, donor organ cells, microvascular cells, cell-free DNA (cfDNA), cell-free RNA (cfRNA), exosomes, proteins, and any combination thereof. In various aspects, the blood and SDF biomarkers used in the disclosed systems and methods may be selected based on the specific indication of a patient, surgery type, surgery site, or any other criterion without limitation.

In various aspects, the blood-based and SDF-based biomarkers may be detected and quantified using any suitable method without limitation. Non-limiting examples of suitable assays for measuring and quantifying the blood-based and SDF-based biomarkers include whole genome sequencing, next generation DNA sequencing, next generation RNA sequencing, PCR, ddPCR, Western blot targeted capture, multiplex PCR, methylation & 16S droplet PCR, proteomics, and any combination thereof. At least a portion of the biomarkers detected and quantified using the disclosed systems and methods are associated with various sources related to surgical wounds, and are indicative of one or more aspects of the systemic condition and/or local wound environment. Non-limiting examples of biomarker sources related to surgical wounds include tumor cells, immune cells, bacterial cells, viral host cells, donor organ cells, microvascular cells, cell-free DNA (cfDNA), cell-free RNA (cfRNA), exosomes, and any combination thereof.

In various aspects, the nucleic acids that are quantified by the disclosed assays are indicative of a variety of different aspects of the local wound environment associated with a surgical procedure. In these aspects, the systems and methods disclosed herein are suitable for use in conjunction with a variety of different surgical procedures including, but not limited to, resectioning surgery, dissection surgery, excision surgery, transplant surgery, reconstructive surgery, and any other suitable surgery type without limitation. In various aspects, a practitioner or surgeon may select any combination of a plurality of assays without limitation. In some aspects, a practitioner or surgeon may order specific or customized assays tailored to a specific indication, surgery type, surgery site, or any other criterion without limitation.

In various aspects, the assays are configured for use with surgical drain fluid as well as blood or plasma. In some aspects, each assay may analyze a separately obtained surgical drain fluid sample. In other aspects, a single surgical fluid sample may be obtained and subjected to analysis by multiple assays. In other additional aspects, a single surgical fluid sample may be obtained and analyzed by all selected assays.

In various aspects, the disclosed assays are configured for use with surgical drain fluid and blood samples. In some aspects, at least a portion of the assays may be based on corresponding blood, plasma, urine, or other fluid sample assays that are modified to render the assay compatible with surgical drain fluid samples. Non-limiting examples of corresponding assays suitable for modification for use with surgical drain fluid samples include liquid biopsy assays such as NavDx™ (Naveris, Natick, Mass., USA) used for the detection of circulating tumor DNA (ctDNA) and any other suitable corresponding assay without limitation. In other aspects, at least a portion of the assays may be developed de novo for use with the surgical drain fluid sample.

Non-limiting examples of suitable assays that may be modified to produce one or more of the assays suitable for the analysis of the surgical drain fluid samples as described herein are described in Molecular Diagnosis and Therapy (2021) 25:757-774, the content of which is incorporated by reference in its entirety.

In some aspects, the disclosed systems and methods enable the collection, preservation, and quantification of cells, proteins, and/or nucleic acids/cfDNA from blood and surgical drain fluid (SDF) samples as early measures of systemic and/or locoregional conditions within a patient following surgery. Non-limiting examples of representative systemic and locoregional conditions of the patient include residual cancer, systemic and/or tumor immunity, wound infection, transplant rejection, microvascular free flap failure, tissue necrosis, and any combination thereof.

In some aspects, the disclosed systems and methods may detect and quantify tumor-associated genetic material and/or proteins including, but is not limited to, cell-free DNA, RNA, proteins, exosomes, and any combination thereof. In other aspects, the tumor-associated genetic material is produced by or associated with a plurality of cancer cells. Non-limiting examples of tumor-associated genetic materials include mutations, overexpression, and underexpression of genes associated with cancer cells. Non-limiting examples of cancer cells include oropharyngeal cancer cells, lung cancer cells, breast cancer cells, melanoma cells, colon cancer cells, thyroid cancer cells, prostate cancer cells, ovarian cancer cells, testicular cancer cells, penile cancer cells, cervical cancer cells, anal cancer cells, brain cancer cells, liver cancer cells, pancreatic cancer cells, and testicular cancer cells.

Additional non-limiting examples of cancer cells include cells from a variety of cancer types including Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

In other aspects, the disclosed systems and methods may detect and quantify additional genetic material indicative of a prognosis or a recommended additional post-surgical treatment. In some aspects, as described in the examples, HPV DNA may be measured and quantified within drain fluid samples and plasma samples collected following neck dissection surgery. The presence or absence of residual HPV DNA after surgery may be used as a locoproximal liquid biomarker to guide the selection of postoperative radiation therapy or chemotherapy in the setting of treatment de-intensification.

In some aspects, the disclosed systems and methods may detect and quantify immune response-related nucleic acids and/or proteins indicative of a local or systemic immune environment within the surgical site. The disclosed systems and methods may detect and quantify cell-free DNA, RNA, proteins, exosomes, and any combination thereof. In other aspects, the immune response-related nucleic acids and/or proteins may be produced by or associated with a plurality of immune cells. Non-limiting examples of immune response-related genetic material and proteins include genes encoding cellular markers associated with immune cells, overexpression or underexpression of genes encoding cytokines or other molecules indicative of an immune environment, and cytokines or other molecules indicative of an immune environment. The immune response-related genetic material and proteins may provide systemic and/or locoproximal measures of immune cell activity and may serve to define a prognosis, to characterize the locoproximal and/or systemic immune environment, and/or may provide information useful in the evaluation of a patient's response to immunotherapy.

In some aspects, the disclosed systems and methods may detect and quantify infection-associated nucleic acids and/or proteins indicative of a locoproximal and/or systemic infection. In various aspects, the infection-associated genetic material detected and quantified includes, but is not limited to, microbe nucleic acids as early markers of systemic infection, wound or surgical site infection, or fistula. In various aspects, the disclosed systems and methods may perform whole genome sequencing of bacteria, fungi, and viruses, as well as targeted capture, multiplex PCR, methylation & 16S droplet PCR. The infection-associated nucleic acids and/or proteins obtained by the disclosed systems and methods may be used to select appropriately targeted antibiotic compositions and doses more quickly and more precisely than wound culture. In other aspects, monitoring of infection-associated nucleic acids and/or proteins may be used to monitor the efficacy of antibiotic treatment.

In some aspects, the disclosed systems and methods may detect and quantify transplant organ nucleic acids indicative of a rejection of a transplant organ by the subject. In various aspects, transplant organ nucleic acids detected and quantified include cfDNA produced by the donor organ. In additional aspects, the disclosed systems and methods may detect overexpression or underexpression of genes encoding biomarkers such as cytokines indicative of acute or chronic transplant organ rejection.

In some aspects, the disclosed systems and methods may detect and quantify flap failure-related nucleic acids and/or proteins indicative of early microvascular free flap failure, necrosis, and any combination thereof. In various aspects, flap failure-related nucleic acids and/or proteins include genes encoding biomarkers for early microvascular free flap failure or necrosis. Non-limiting examples of suitable biomarkers for early microvascular free flap failure or necrosis include cfDNA concentrations of the genes Prol1, Muc1, Fcnb, Il 1b, and/or Vcsa1.

### I. Methods of Detecting Surgery-Related Conditions

In various aspects, the systems and methods described herein may be used in a method for detecting systemic conditions and locoproximal conditions within a surgical site of a subject based on the analysis of blood and surgical drainage fluid (SDF) samples obtained following a surgery. As described above, the systemic and locoproximal conditions monitored using the method described herein include, but are not limited to, molecular margin or minimal residual cancer, local and systemic immune environment, local and systemic infection, rejection or failure of a transplant organ, early microvascular free flap failure or necrosis, and any other relevant surgery-related condition without limitation.

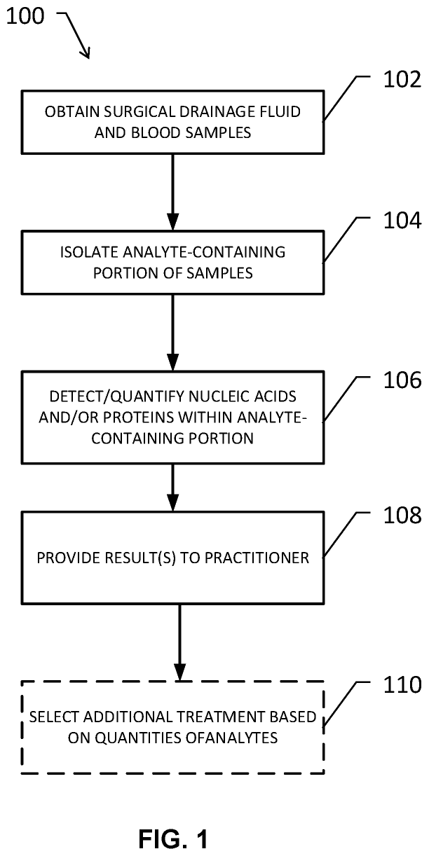

FIG. 1 is a flow chart illustrating the steps of a method 100 for detecting surgery-related systemic and locoproximal conditions in one aspect. The disclosed method 100 makes use of nucleic acid and/or protein assays configured to detect analytes within a patient's blood and surgical drainage fluid samples. A practitioner or surgeon may select the nucleic acid and/or protein assays used in the method 100 based on the type of surgery, the disorder treated by the surgery, anticipated risk factors such as infection or microvascular failures, anticipated post-surgical treatments, and any other suitable criterion without limitation.

Referring to FIG. 1, the disclosed method 100 includes obtaining a surgical drainage fluid and blood samples from the patient at 102. In various aspects, the blood sample may be obtained from the patient using any suitable method without limitation including a venous draw. In various other aspects, the surgical drain fluid samples may be obtained using any suitable surgical drain device associated with any type of surgical procedure without limitation. In some aspects, the surgical drain fluid sample may be obtained using a surgical drain tube, a surgical wound vac, and any other suitable surgical drainage device without limitation. In other aspects, the surgical drain fluid samples may be obtained using custom surgical drain devices specifically provided with elements configured to preserve the integrity of nucleic acids, proteins, and other analytes within the sample, to perform at least a portion of sample preparation steps, and any other suitable function related to obtaining, preserving, and processing a surgical fluid sample for analysis without limitation. By way of non-limiting example, the surgical drainage sample may be obtained using a surgical drain configured to collect and preserve nucleic acid biomarkers as described in PCT Application PCT/US2022/020889, the content of which is incorporated herein by reference in its entirety.

In some aspects, the blood and SDF samples may be obtained within about 24 hours of the completion of the surgery, providing the practitioner with timely information regarding genetic materials, proteins, and/or other analytes in the surgical drainage that may be used to select additional treatments. In various other aspects, the sample may be obtained within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 24 hours, about 32 hours, about 36 hours, about 48 hours, or about 72 hours of the completion of the surgery.

In other aspects, blood and SDF are sampled two or more times at different times following surgery to monitor systemic and locoproximal conditions over time. In one aspect, may be obtained, a blood sample may be obtained prior to the surgery and analyzed as described herein to serve as a reference.

Referring again to FIG. 1, the disclosed method 100 further includes isolating portions of the blood and surgical drain fluid samples containing nucleic acids, proteins, and other analytes as described above for analysis at 104. In various aspects, the isolation of the nucleic acids and other analytes is performed in a manner that preserves sufficient integrity of these compounds for detection by the assays as described herein. The portions of the blood and surgical drain fluid samples containing nucleic acids, proteins, and other analytes may be isolated from the samples using any suitable method without limitation. Non-limiting examples of suitable isolation methods include filtering the samples, centrifuging the samples, contacting the samples with a chromatography medium, and any combination thereof.

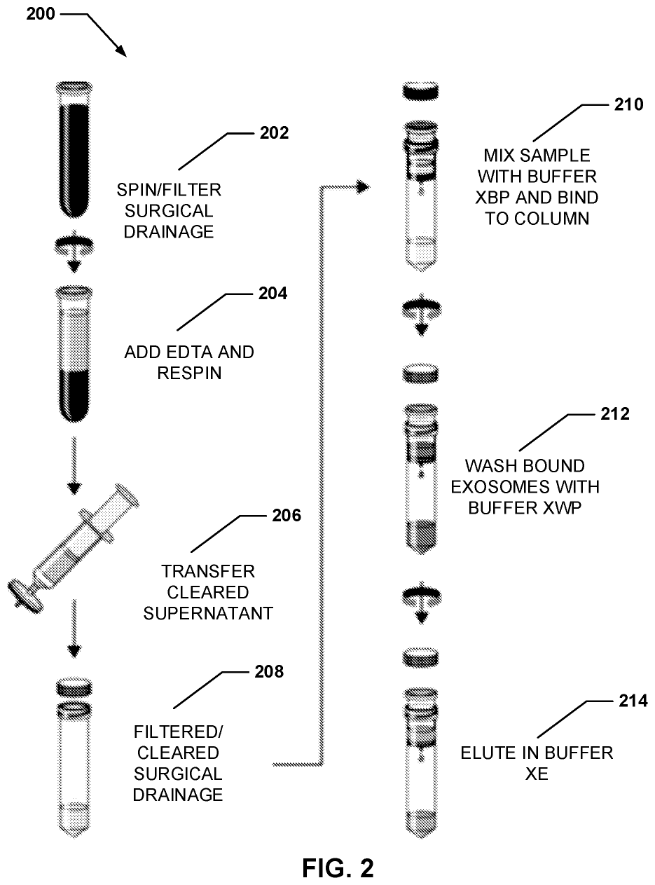

By way of non-limiting example, FIG. 2 is a schematic illustration of a method 200 of isolating the tumor-associated genetic material from the sample in one aspect. As illustrated in FIG. 2, the surgical drainage is centrifuged and filtered at 202. EDTA is added to the sample to inhibit nucleases in the sample and the sample with added EDTA is further centrifuged at 204. The supernatant is removed from the surgical drainage mixture at 206 and retained. In some aspects, the supernatant may be used as-is for the detection and quantification of cell-free DNA, RNA, and proteins. In other aspects, the supernatant may be filtered and cleared at 208 prior to further treatment as illustrated in FIG. 2.

In other aspects, illustrated in FIG. 2, exosomes may be isolated from the supernatant by contacting the supernatant with chromatographic media followed by elution. As illustrated in FIG. 2, the filtered and cleared sample from 208 may be mixed with buffer XBP and bound to a column at 210. The column-bound exosomes may be washed with buffer WP at 212 and eluted from the column with buffer XE at 214. In various aspects, the isolation of the tumor-associated genetic material using the method illustrated in FIG. 2 is configured to yield intact exosomes.

Referring again to FIG. 1, the method further includes detecting and quantifying nucleic acids, proteins, and other analytes within the isolated portions of the blood and surgical drain fluid samples at 106. Any suitable method may be used to sequence, detect, and quantify the tumor-associated mutations and/or variants including, but not limited to, next generation DNA sequencing, next generation RNA sequencing, next generation protein sequencing, PCR, Western blot, and any combination thereof. In some aspects, a targeted sequence assay panel may be used.

Referring again to FIG. 1, the method may further include providing the assay results including, but not limited to, quantities of detected proteins, nucleic acid mutations, variants, and/or over/underexpressions to a practitioner at 108. In some aspects, the assay results may include individual quantities and/or expression profiles of nucleic acids of interest. In other aspects, assay results may be provided in the form of one or more summary scores obtained by comparing the characteristics of the detected nucleic acids or other analytes to previously-obtained criteria indicative of a surgery-related condition, a prognosis, and/or a recommendation for a post-surgical treatment as described above. In some aspects, an individual summary score may be reported for each selected assay module. In other aspects, the results of two or more assay modules may be combined to produce an overall summary score. In these other aspects, the overall summary score may be based on a mathematic combination of the results of the two or more assay modules including, but not limited to, sums, differences, products, ratios, minima, maxima, means/averages, correlation coefficients, any other suitable mathematical relationship, and any combination thereof.

In various aspects, the method optionally includes selecting at least one additional or adjuvant treatment based on the quantities of SDF and plasma analytes at 110. Based on the detected quantities or results of the selected modular assays, the practitioner or surgeon may make a determination of a post-surgical condition or prognosis in the patient, and/or select a follow-up treatment. Non-limiting examples of suitable follow-up treatments include radiotherapy, chemotherapy, follow-up surgery, active surveillance with imaging, antibiotic treatment, and any combination thereof.

In some aspects, the systemic and/or locoproximal biomarkers obtained using the methods disclosed herein may be used to produce time to recurrence assessments using Cox regression and Kaplan-Meier analysis. In other aspects, the disclosed method may implement WGS using ichorCNA (Broad) to infer tumor fraction in drain fluid; previously ichorCNA has only been used in plasma. A higher tumor fraction is likely to correlate with high-risk pathology and elevated HPV copy number. In some aspects, chimeric reads can be used to determine integration status. In another additional aspect, whole-exome sequencing (WES) may be used to infer tumor mutational burden. The disclosed method has to potential to pave the path toward personalized adjuvant immunotherapy in the future. In some aspects, the disclosed method may implement HPV capture sequencing on suspected false-negative cases (high-risk pathology, early recurrence, but no HPV copies detected in drain fluid). One non-limiting example of false-negative cases that may benefit from further analysis using the disclosed method includes a patient group such as 30L (ENE+, received CRT and recurred, HPV copies=ND). In some aspects, the disclosed method may be used to implement T cell receptor profiling (Adaptive immunoSEQ) to examine T cell diversity in drain fluid, and track clones in subsequent plasma timepoints that expand with radiation through DAMP response potentiation. In some aspects, the disclosed method may be implemented using cell-free RNA with CibersortX to assess T cell clonality in SDF.

In various aspects, the disclosed method provides for the sequencing or molecular measures of a variety of systemic and locoproximal surgery-related conditions including, but not limited to, residual cancer, infection, immune environment, risk of poor wound healing, and transplant organ rejection using the analysis of blood and surgical drain fluid samples.

### EXAMPLES

The following examples are provided to illustrate various aspects of the disclosure.

### Example 1: Surgical Drain Fluid Liquid Biopsy Analysis of Locoregional Residual Disease after Surgery in HPV+ Oropharyngeal Cancer for Adjuvant Radiotherapy Risk Stratification

To evaluate the method of evaluating a patient's condition following surgery by analysis of surgical drain fluid (SDF) described herein, the following experiments were conducted.

In the current era of treatment de-intensification for HPV+ oropharyngeal squamous cell carcinoma (OPSCC), risk stratification for adjuvant treatments after surgery is needed to avoid the overuse of radiation therapy or combinatorial therapies after surgery. RT side effects include pharyngeal constriction, glossitis, and dysphagia leading to anorexia. Risk stratification is especially important in HPV+OP SCC to assure favorable outcomes.

Since biomarkers are not typically used to personalize adjuvant treatments, side effects may needlessly decrease quality of life. In the experiments described below, the assessment of biomarkers within surgical drain fluid (SDF) was assessed as an objective, locoproximal, and quantifiable biomarker of locoregional residual disease (LRD). It was hypothesized that elevated HPV viral load in SDF correlated directly with high-risk surgical pathology. It was also hypothesized that post-operative residual HPV in SDF might represent local residual disease due to occult cancer cells releasing fragments of HPV DNA in the lymph fluid. Post-operative measurements of HPV viral loads in SDF may help risk-stratify adjuvant chemo/radiotherapy.

Eighty-four SDF neck dissection specimens were collected postoperatively at 24 hours from 58 HPV+ OPSCC patients. Inclusion criteria included patients greater than 18 years with a diagnosis of HPV+ OPSCC (p16+by IHC) undergoing resection of the primary tumor with neck dissection with postoperative drainage. Exclusion criteria included patients on whom a lymph node biopsy was performed, having surgery performed for recurrent disease, and/or patients that were previously denied primary tumor surgery. SDF blood content was measured using the NanoDrop Oxy-hemoglobin method.

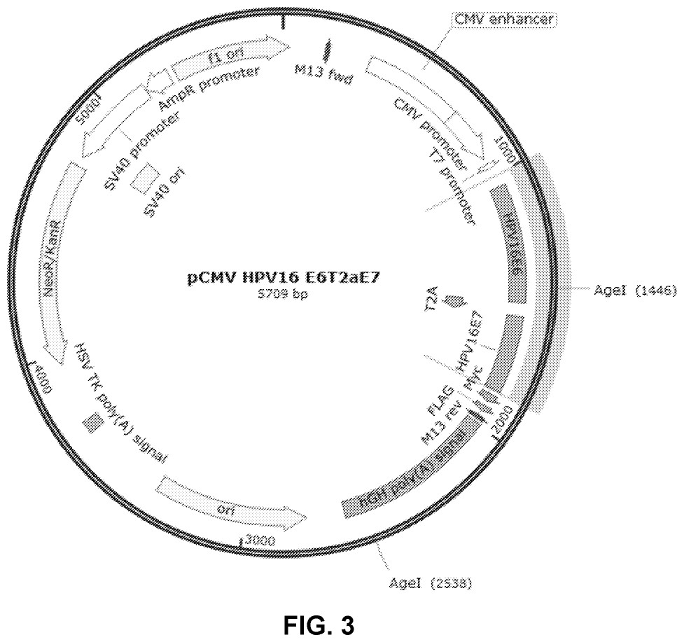

The limit of detection (LOD) of Taqman quantitative PCR (qPCR) was compared to digital droplet PCR (ddPCR) by analyzing 10-fold dilutions of the HPV16 E6T2aE7 plasmid; the E6T2aE7 plasmid is illustrated schematically in FIG. 3. The moles and copy numbers of measured dsDNA were determined as expressed below:

$$ds DNA ( mol ) = \frac{( mass of ds DNA ( g ) )}{( ( length of ds DNA ( bp ) \times 617.96 g {mol}^{- 1} b {p}^{- 1} ) + 36.04 g / mol )} \times 2$$

$$copy number = ds DNA ( mol ) \times 6.02 {2}^{23}$$

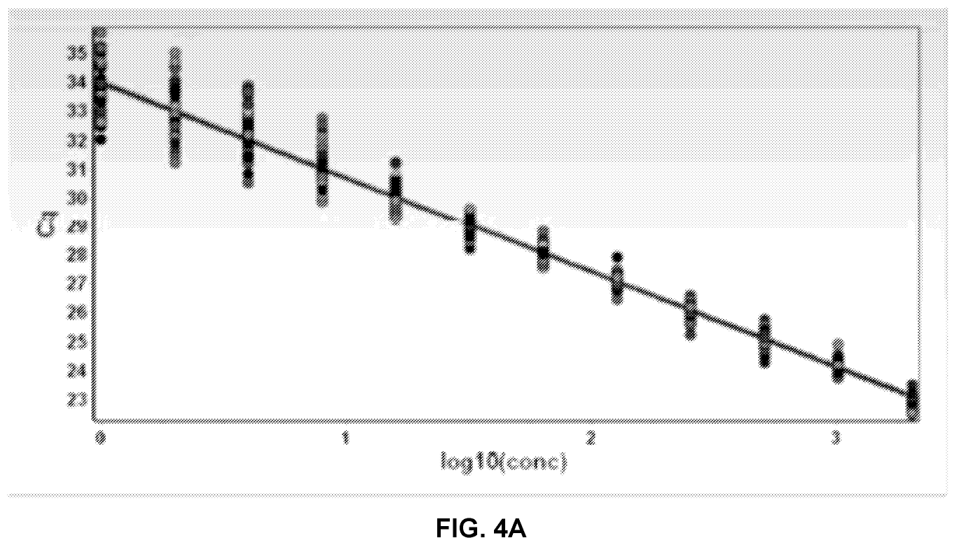

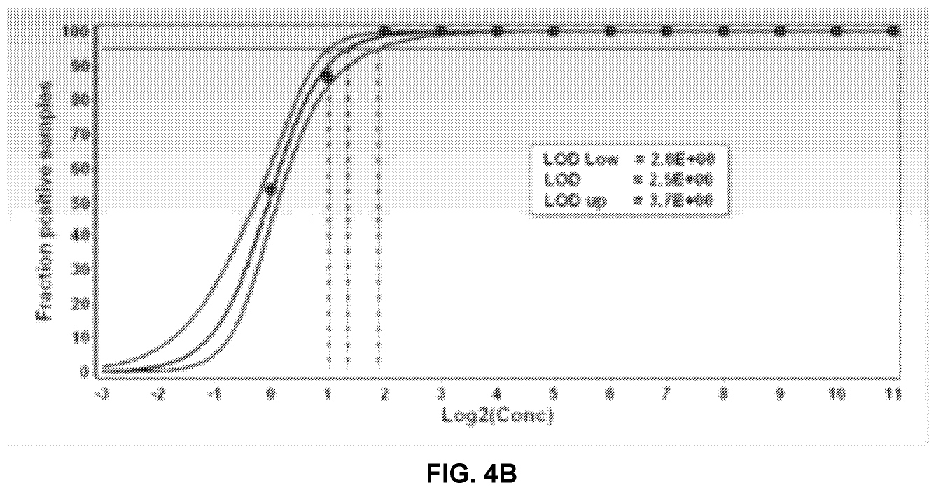

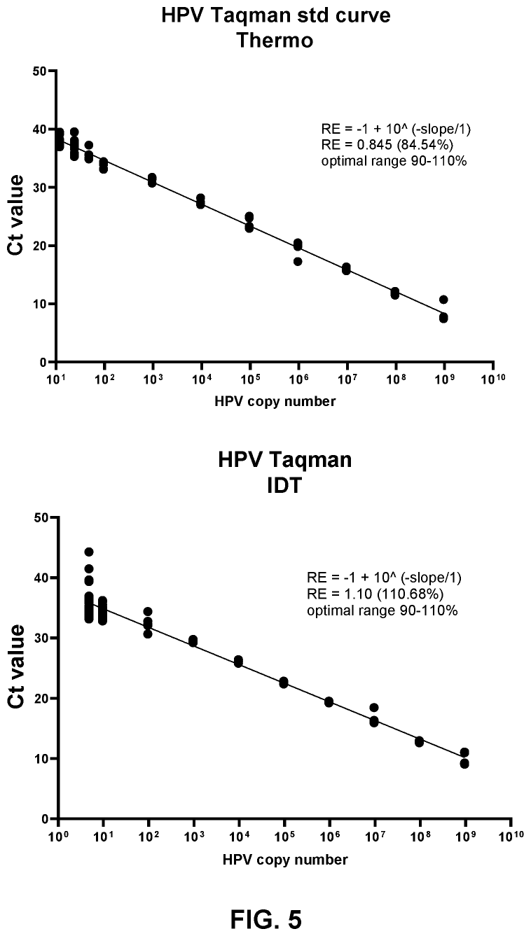

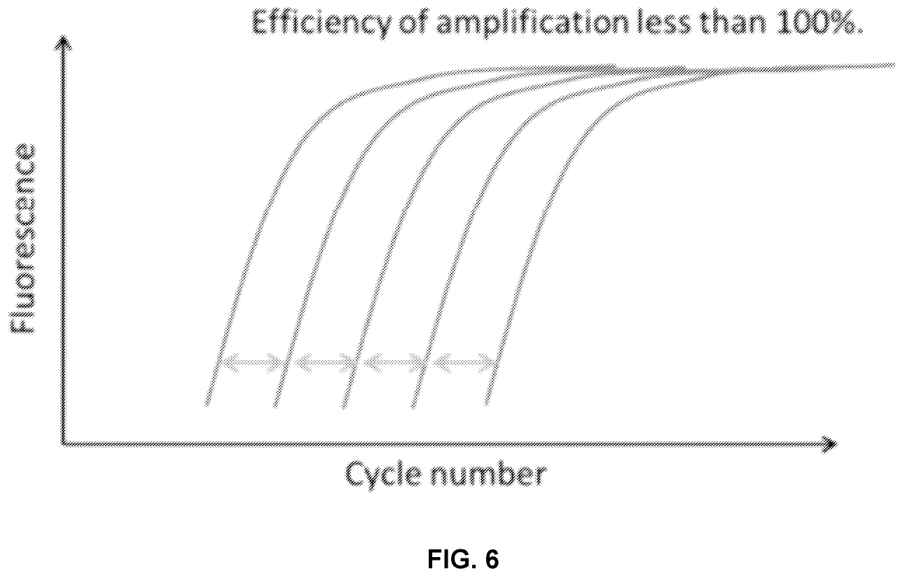

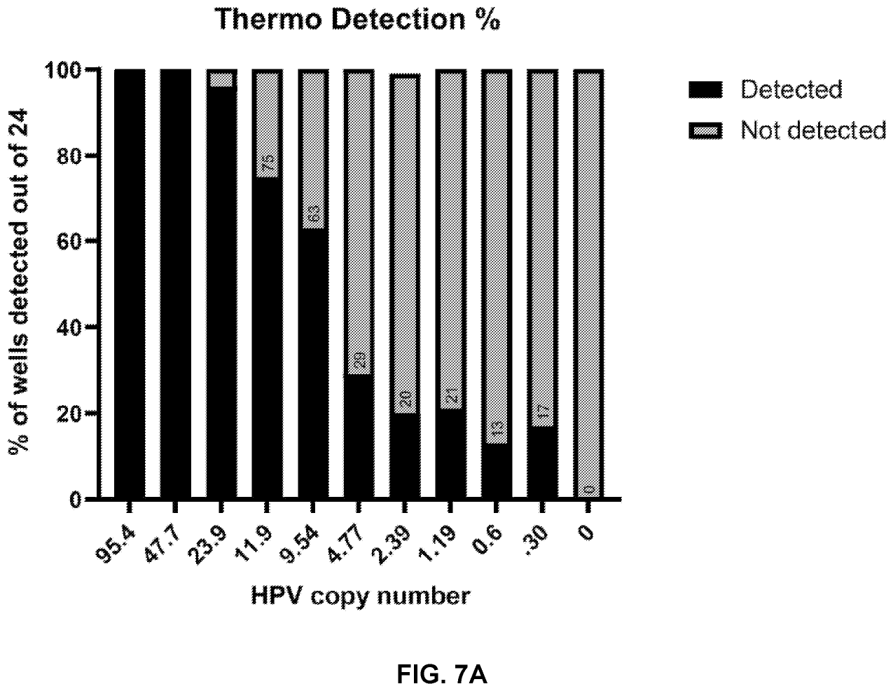

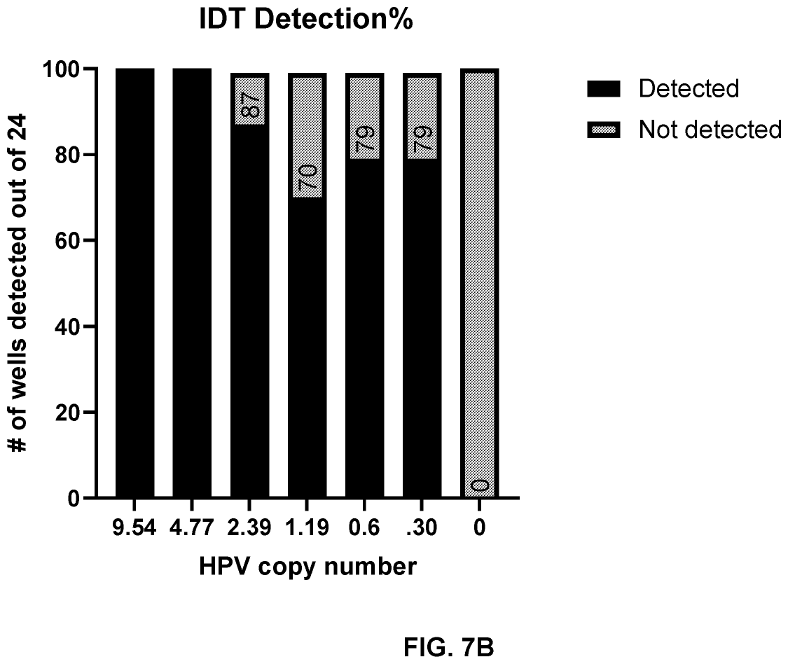

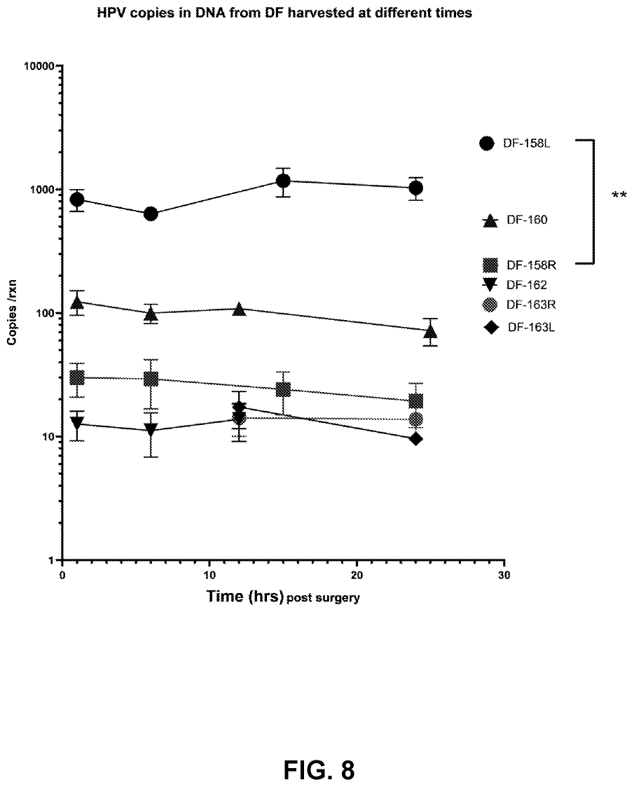

A standard curve was generated using a method that would allow the assay's limit of detection to be established (FIGS. 4A, 4B, 5, and 6). The level of detection (LOD) was determined to be 4.7 copies using both Thermo (FIG. 7A) and IDT (FIG. 7B) master mix reagents (24 replicates, >95% confidence). Measurements were stable over 24 hours (FIG. 8). HPV copies/uL in DNA eluted from SDF were then compared to pathological features (ENE, number of positive nodes). HPV detection was also performed in paired plasma samples from 25 node+ patients. Statistical analyses included Wilcoxon, Kruskal-Wallis, Fisher's exact testing, and Spearman correlation. AJCC 8th edition was used for staging.

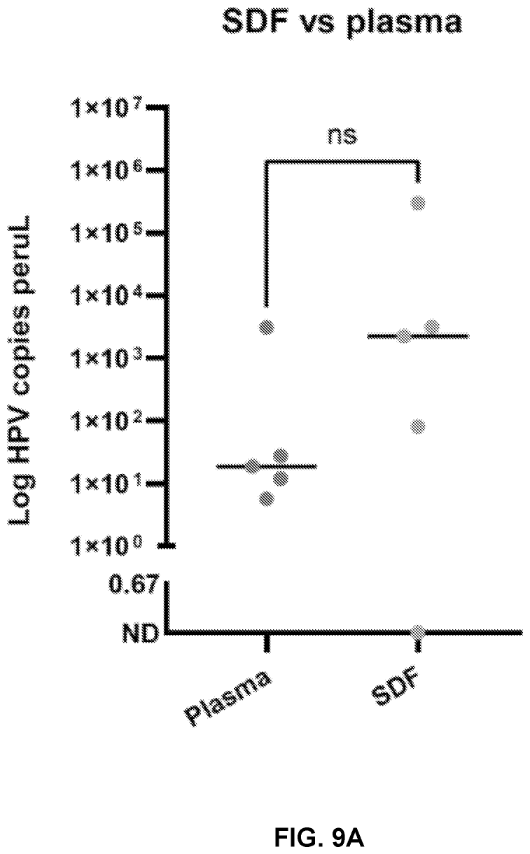

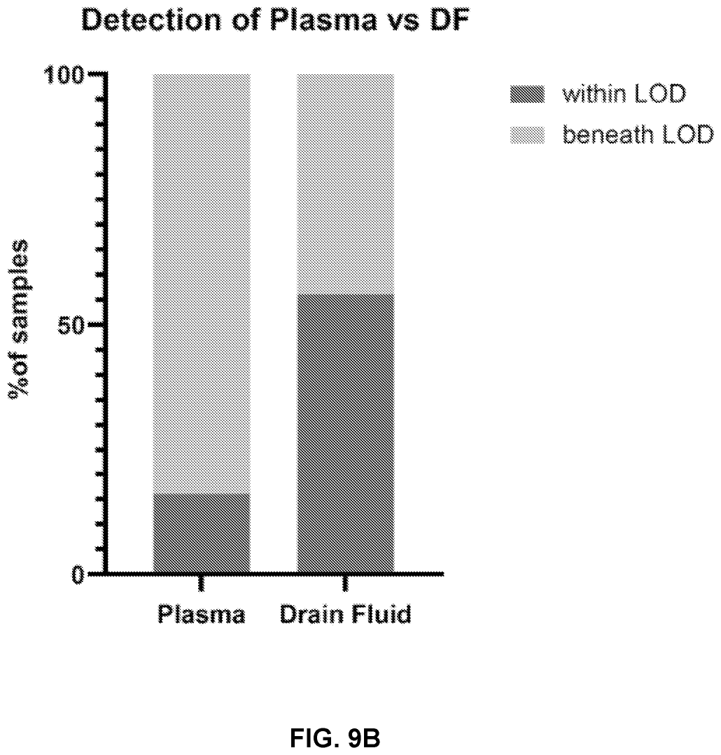

Experiments were performed to measure HPV levels in surgical drainage fluid (SDF) and plasma of the patients described above. SDF HPV copies were measured at relatively higher (non-significant) levels than corresponding plasma-measured HPV, both in terms of HPV copies (FIG. 9A) and as a percent of samples (FIG. 9B); the vast majority of plasma HPV was undetectable at this timepoint.

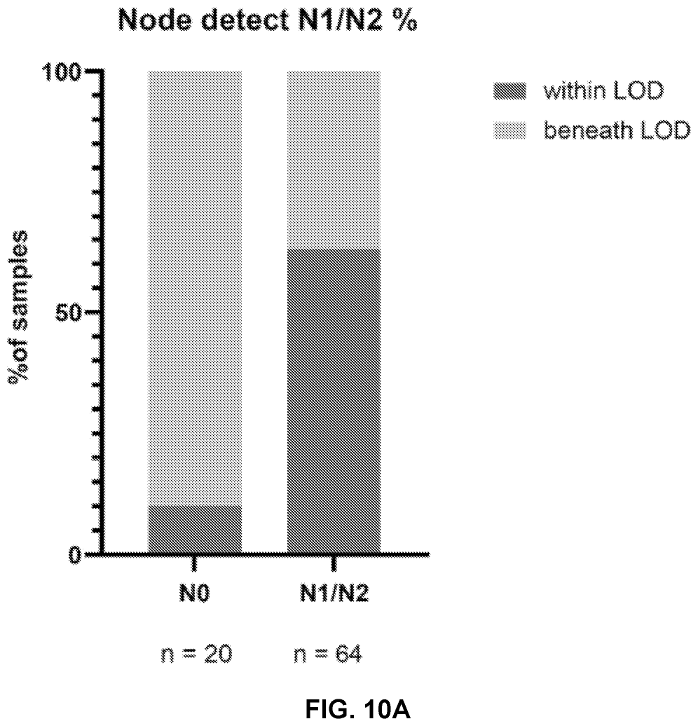

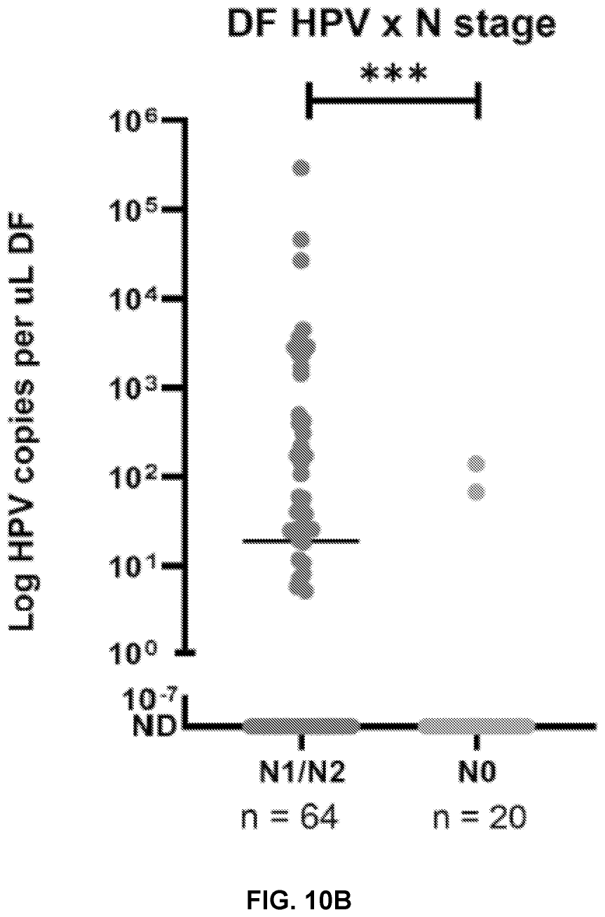

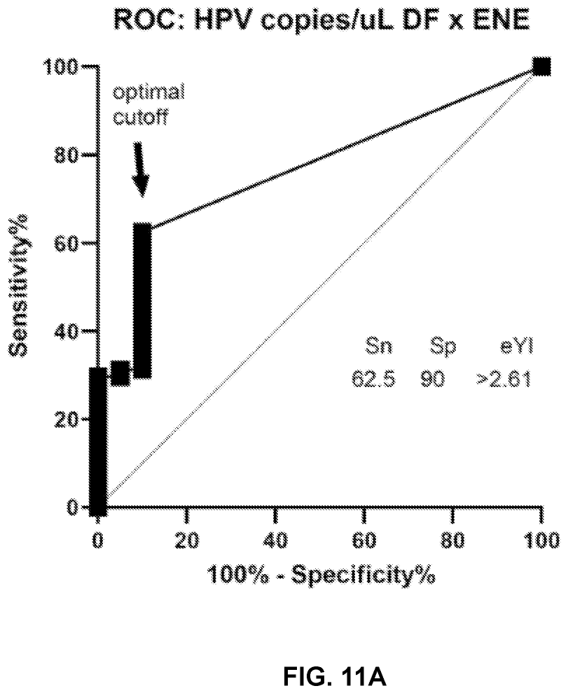

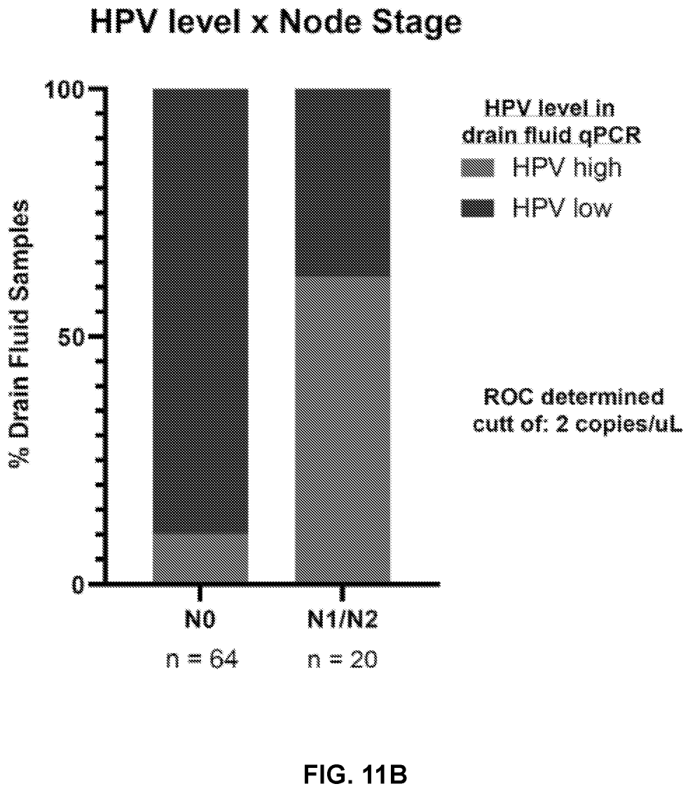

Measured HPV levels were compared in patients with N0 and N1/N2 graded lymph nodes. More patients graded with N1/N2 lymph nodes had detectable levels of HPV compared to those graded with N0 lymph nodes (FIG. 10A). The levels of HPV in drainage fluid from patients with N1/N2 graded nodes were higher than comparable HPV levels in drainage fluid from patients with N0 nodes (FIG. 10B). Further, the collection of drainage fluid samples from N1/2 nodes resulted in more samples with high HPV amounts than comparable samples from N0 nodes (FIGS. 11A and 11B).

Hemoglobin concentration (data not presented) had no correlation to HPV levels (p=−0.07, p=0.53).

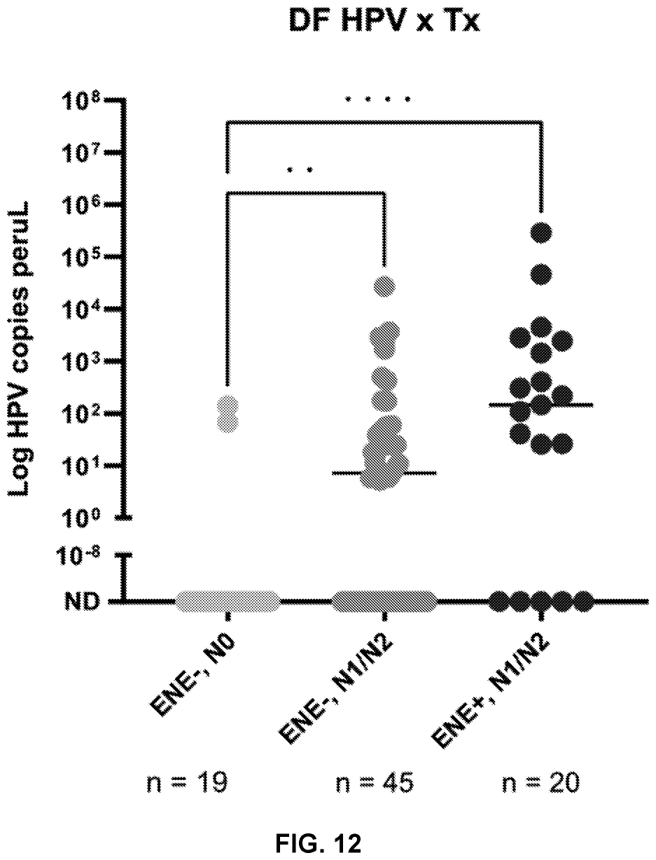

As illustrated in FIG. 12, the proportion of patients with nodes graded N1/N2 and HPV levels above LOD was 6-fold higher than the comparable proportion of patients with nodes graded N0 (p<0.0001). Furthermore, median HPV copies were 20-fold higher in ENE+N1/N2 patients compared to ENE−N1/N2 patients (p=0.015; FIG. 12), suggesting that elevated post-operative SDF HPV detection levels were indicative of invasive nodal disease and served as a proxy for locoregional residual disease (LRD) obtained using surgical pathology.

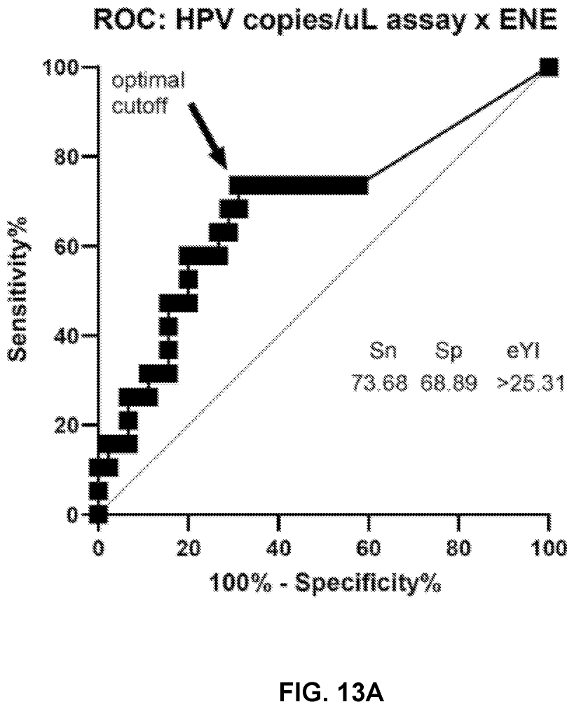

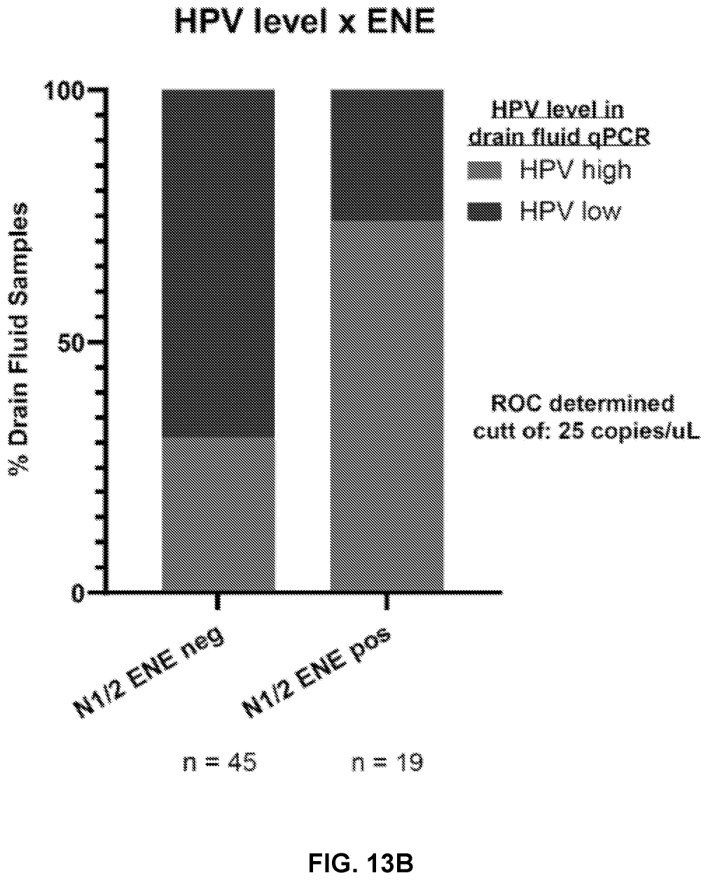

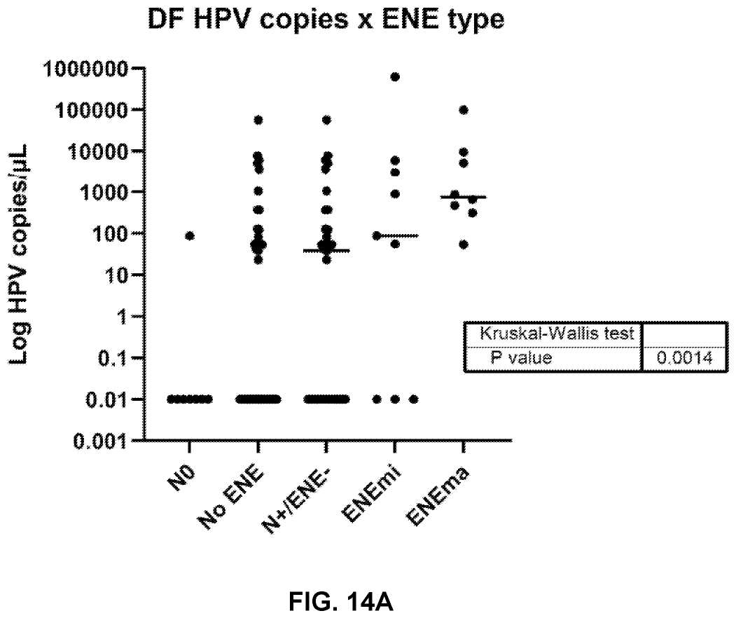

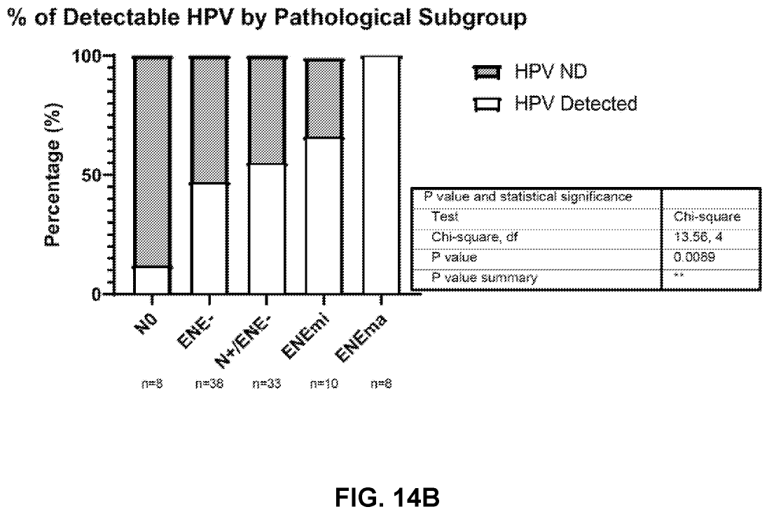

Surgical drain fluid (SDF) analysis was demonstrated to predict ENE status in a surgical specimen, as more samples from N1/2 ENE positive nodes contained high amounts of HPV copies compared to N1/2 ENE negative nodes (FIGS. 13A and 13B). Further, the number of HPV copies detected in SDF was dependent on ENE status, as illustrated in FIGS. 14A and 14B.

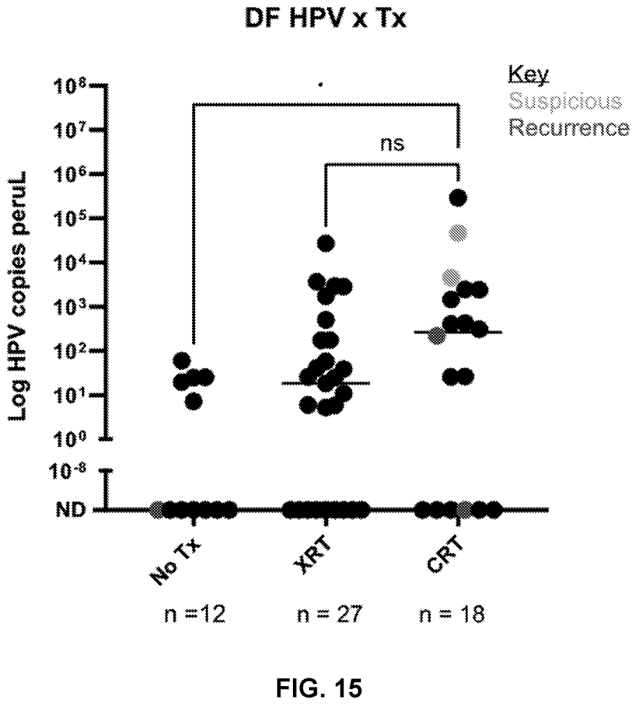

To assess whether adjuvant therapy escalation occurred in patients with higher-risk diseases, SDF HPV levels were stratified by eventual adjuvant therapy type—radiation alone (XRT), chemoradiation (CRT), or none (No Tx), as illustrated in FIG. 15. Median HPV load in SDF was significantly higher in CRT and XRT relative to the No Tx cohort (p=0.044; FIG. 15). Strikingly, 15 (33%) patients treated with XRT or CRT had undetectable HPV in their SDF, suggesting that these patients could potentially have safely undergone treatment de-escalation. In addition, 3 of 18 (17%) patients treated with adjuvant CRT with detectable HPV in SDF experienced suspicious or confirmed recurrence, suggesting they could have potentially benefited from further or orthogonal treatment escalation (i.e. immunotherapy). Lastly, only 4 (16%) post-operative paired plasma samples had detectable HPV, implying inferior sensitivity for detecting LRD compared to HPV levels detected in SDF.

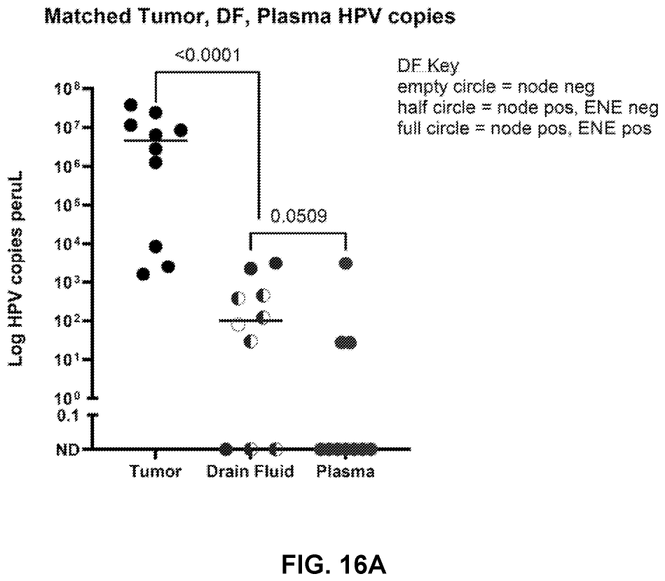

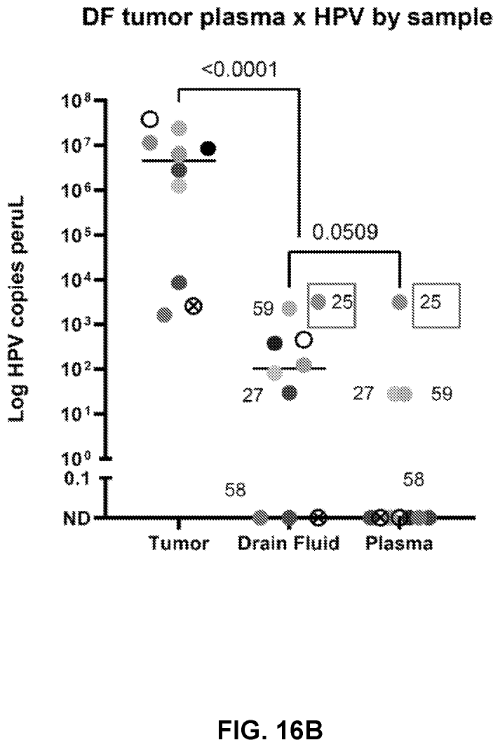

To assess the relative abundance of HPV copies in different samples, HPV levels were measured in matched samples of tumor tissue, SDF, and plasma, as summarized in FIGS. 16A and 16B. The numbers of HPV copies measured in tumor tissue samples were higher than in matched SDF samples (p<0.0001). HPV copies detected in SDF samples were higher than HPV levels detected in plasma samples (p=0.0509).

The results of these experiments demonstrated that postoperative qPCR of SDF enabled LRD detection in HPV+ OPSCC patients. SDF was highly enriched for HPV compared to plasma and correlated significantly with aggressive pathologic features (nodal stage, ENE) as well as adjuvant treatment escalation. Postoperative SDF analysis may supplement traditional pathology methods to implement personalized adjuvant therapy for surgically treated HPV+ OPSCC patients.

### Example 2: Combined Analysis of Blood and Surgical Drain Fluid To Assess Locoregional Residual Disease and Metastasis After Surgery in HPV+ Oropharyngeal Cancer

To evaluate a method of evaluating a patient's condition following surgery by analysis of blood plasma and surgical drain fluid (SDF), the following experiments were conducted.

The long-term outcomes of selected patients from the population described in Example 1 were reviewed to assess the predictive efficacy of the method of blood/SDF biomarkers obtained as disclosed. A summary of the patients reviewed, each patient's risk path as assessed by conventional post-surgical imaging, as well as each patient's levels of HPV copies in post-surgical plasma and SDF samples are summarized in Table 1 below:

TABLE 1
Patients Reviewed for Long-Term Outcomes

| | | SDF HPV | Plasma HPV |
|---|---|---|---|
| Patient | Risk Path | Copies | Copies |
| DF025 | High | Elevated | Elevated |
| DF027 | Low | Elevated | Elevated |
| DF058 | High | Low | Low |

Patient DF025 was identified as high-risk based on a pre-surgery PET image indicating sub-centimeter pulmonary nodules, as well as the finding of 3.9 cm p16+ SCC with extensive LVI, −PNI, −Margin; 15/134 nodes positive (4.1 cm) with ENE and +margin at jugular foramen. Despite aggressive treatment, metastasis was present in PET and CT images obtained 3 months and 6 months after surgery, and the patient's death occurred 14 months after the initial diagnosis, as predicted by the elevated levels of HPV copies detected in both plasma and SDF samples obtained after surgery. HPV levels in the tumor, drainage fluid, and plasma of high-risk path, plasma/SDF double-positive patients are compared in FIGS. 16A and 16B, with measurements of this patient denoted by patient number (25) in superimposed boxes.

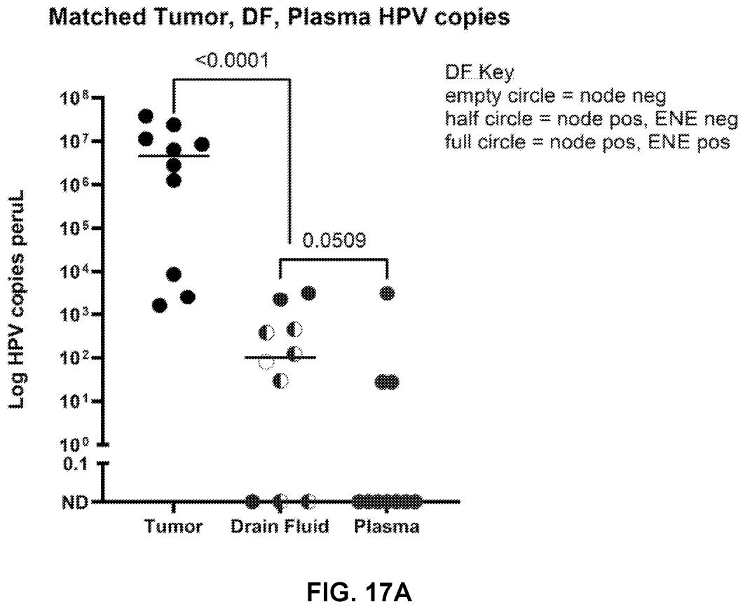

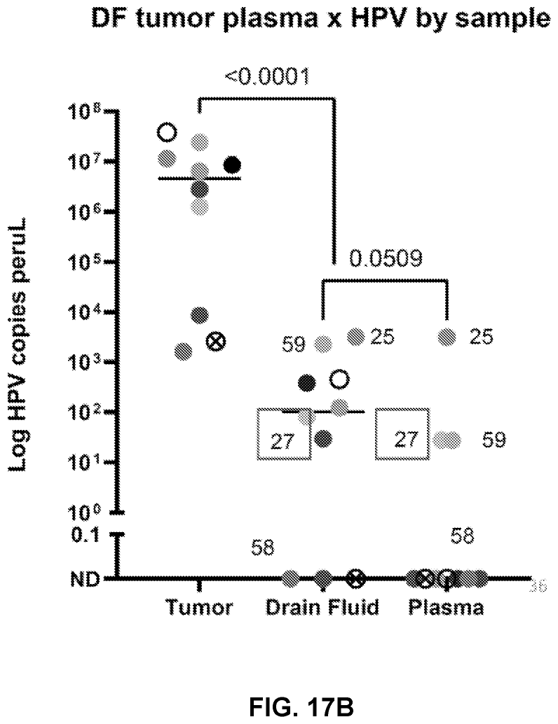

Patient DF027 was identified as low-risk based on images obtained before surgery indicating no LVI, no PNI, 1 positive node (4.3 cm) without ENE, and neg margin. No post-surgical adjuvant radiation treatment or chemoRT was offered to patient DF027 due to the post-surgical finding of ENE & margins negative. A local recurrence in the left neck of the patient occurred 11 months after surgery, as predicted by the elevated plasma and SDF levels of HPV copies. HPV levels in the tumor, drainage fluid, and plasma of high-risk path, plasma/SDF double-positive patients are compared in FIGS. 17A and 17B, with measurements of this patient denoted by patient number (27) in superimposed boxes.

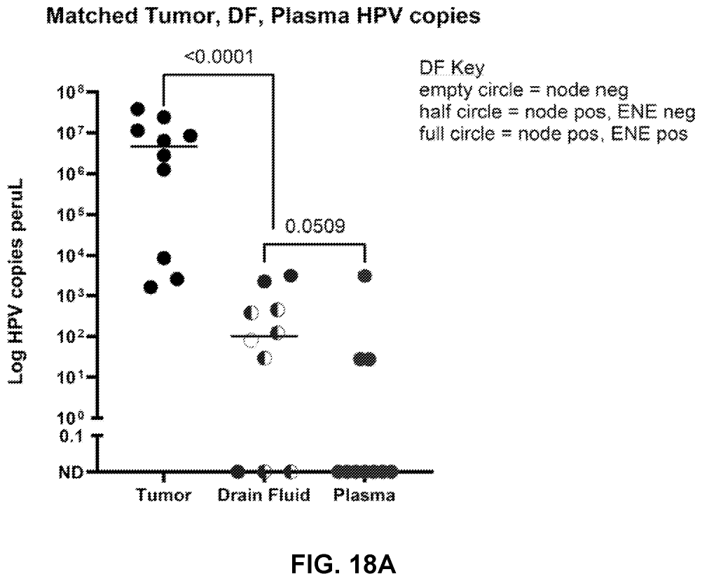

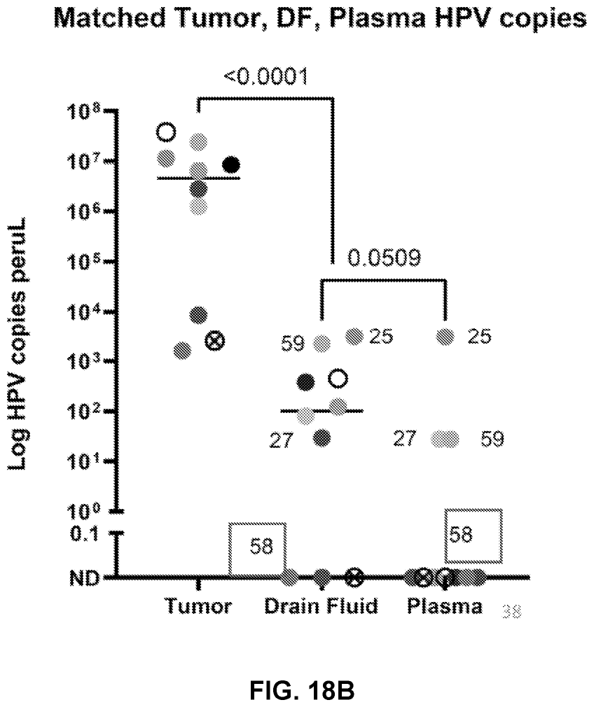

Patient DF058 was identified as high-risk based on images obtained before surgery indicating 4.7 cm p16+ SCC with extensive LVI, −PNI, −Margin; 1/40 (right) node positive (3.8 cm) with ENE and +margin. Adjuvant post-surgical treatment was provided in the form of chemoRT (21 fractions of 42 Gy concurrent w/1 cycle+cisplatin). The chemoRT treatment included reduced dosing and fractionation of the radiation, which is typically administered in 2 cycles of 60 Gy. No recurrence was observed in this patient, as predicted by the low levels of HPV copies detected in both plasma and SDF samples obtained after surgery. HPV levels in the tumor, drainage fluid, and plasma of high-risk path, plasma/SDF double-positive patients are compared in FIGS. 18A and 18B, with measurements of this patient denoted by patient number (58) in superimposed boxes.

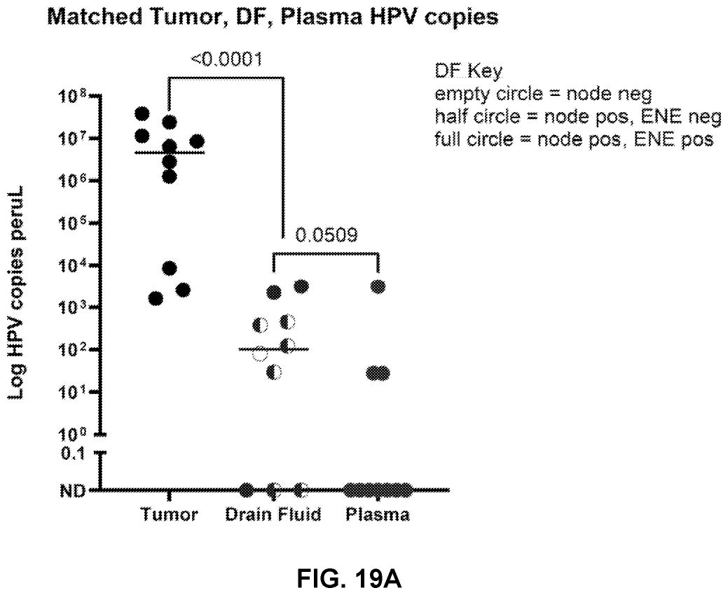

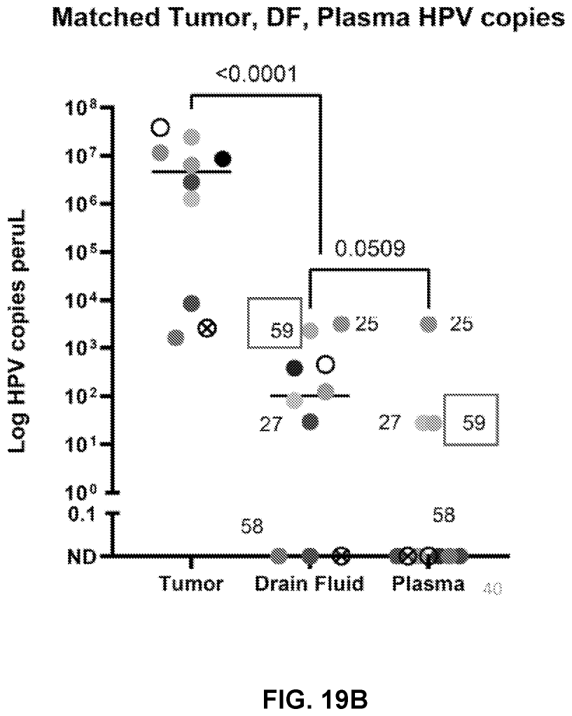

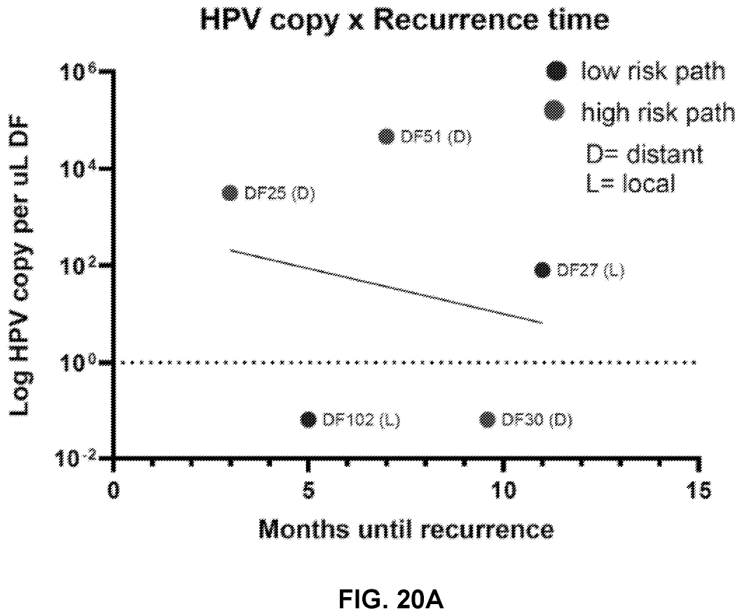

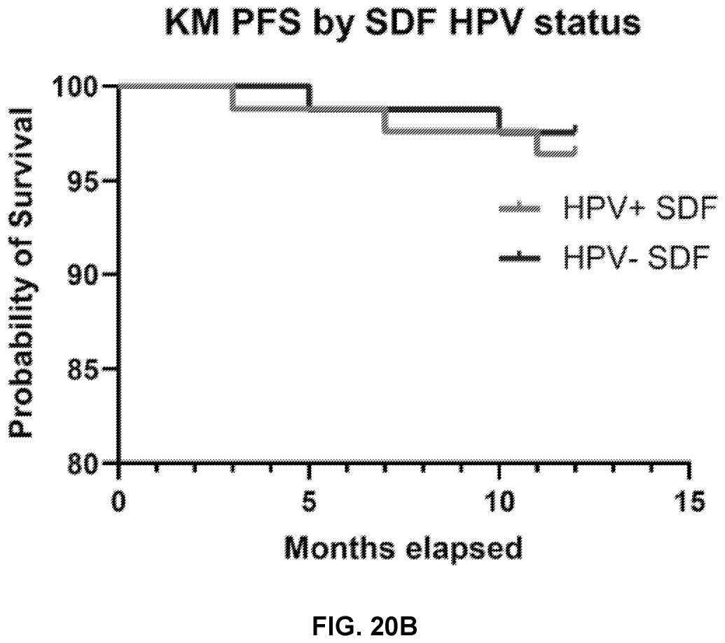

Patient DF059 was identified as high-risk based on images obtained before surgery indicating 2.6 cm p16+ SCC with extensive LVI, −PNI, −Margin; 1 node positive (5 cm) with ENE+. No adjuvant post-surgical treatment was provided as of the time of assessment. A recurrence was predicted to occur at 7 months post-surgery, based on a regression of measurements from 3 relapsed patients with HPV+ SDF (FIG. 20A). HPV levels in the tumor, drainage fluid, and plasma of high-risk path, plasma/SDF double-positive patients are compared in FIGS. 19A and 19B, with measurements of this patient denoted by patient number (59) in superimposed boxes.

The SDF HPV levels obtained from patients with recurring oropharyngeal cancer were analyzed to identify a correlation between HPV levels and months until recurrence. As illustrated in FIG. 20A, a trend was observed, but the sample size was insufficient to infer a significant correlation. A survival curve constructed based on the patient population described above indicated higher survival rates for patients with HPV− SDF samples, as illustrated in FIG. 20B, but again sample size was insufficient to infer a significant correlation.

The results of these experiments demonstrated that the levels of HPV copies in plasma and SDF samples obtained after surgery were predictive of patient prognosis, and could be used to select an appropriate adjuvant post-surgical treatment ranging from de-escalated RadioRT to aggressive RadioRT, depending on the plasma/SDF samples.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

What is claimed is:

1. A method for detecting at least one systemic condition and at least one locoproximal condition within a surgical site of a subject following a surgery, the method comprising:
a. obtaining a surgical drainage fluid sample from the surgical site and a blood sample from the subject;
b. isolating analyte-containing portions from the surgical drainage fluid sample and the blood sample;
c. detecting and quantifying at least a portion of analytes within the analyte-containing portions to produce at least one assay result, wherein the at least one assay result is indicative of the at least one systemic condition and the at least one locoproximal condition within a surgical site of a subject; and
d. providing the at least one assay result to a practitioner.

2. The method of claim 1, wherein the surgery is selected from a resectioning surgery, a dissection surgery, an excision surgery, a transplant surgery, a reconstructive surgery, and any combination thereof.

3. The method of claim 1, wherein the analyte-containing portions isolated from the surgical drainage fluid sample and the blood sample each comprise cfDNA, RNA, exosomes, tumor cells, immune cells, bacterial nucleic acids, viral nucleic acids, proteins, and any combination thereof.

4. The method of claim 1, wherein the analytes within the analyte-containing portions are detected and quantified using at least one assay selected from whole genome sequencing, next generation DNA sequencing, next generation RNA sequencing, PCR, Western blot targeted capture, multiplex PCR, methylation & 16S droplet PCR, and any combination thereof.

5. The method of claim 1, wherein isolating the analyte-containing portions from the surgical drainage fluid sample and the blood sample comprises filtering the samples, centrifuging the samples, contacting the sample with chromatography media, and any combination thereof.

6. The method of claim 1, further comprising selecting an additional treatment based on the at least one assay result.

7. The method of claim 6, wherein the additional treatment is selected from radiotherapy, chemotherapy, follow-up surgery, active surveillance with imaging, antibiotic therapy, antiviral therapy, and any combination thereof.

8. The method of claim 1, wherein obtaining the surgical drainage fluid sample from the subject further comprises capturing surgical drainage fluid from a drainage tube associated with the surgery.

9. The method of claim 1, wherein obtaining the surgical drainage fluid sample from the subject further comprises capturing the surgical drainage fluid from the drainage tube within about 24 hours of the surgery.

10. A method of selecting at least one additional treatment for a subject following a surgery, the method comprising:
a. detecting at least one systemic condition and at least one locoproximal condition of the subject by:
i. obtaining a surgical drainage fluid sample from the surgical site and a blood sample from the subject;
ii. isolating analyte-containing portions from the surgical drainage fluid sample and the blood sample; and
iii. detecting and quantifying at least a portion of analytes within the analyte-containing portions to produce at least one assay result,
wherein the at least one assay result is indicative of the at least one systemic condition and the at least one locoproximal condition within a surgical site of a subject; and
b. selecting the at least one additional treatment based on the at least one systemic condition and at least one locoproximal condition detected in the subject.

11. The method of claim 10, wherein the at least one additional treatment is selected from radiotherapy, chemotherapy, follow-up surgery, active surveillance with imaging, antibiotic therapy, antifungal therapy, and any combination thereof.

12. The method of claim 10, wherein the surgery is selected from a resectioning surgery, a dissection surgery, an excision surgery, a transplant surgery, a reconstructive surgery, and any combination thereof.

13. The method of claim 10, wherein the analyte-containing portions isolated from the surgical drainage fluid sample and the blood sample each comprise cfDNA, RNA, exosomes, tumor cells, immune cells, bacterial nucleic acids, viral nucleic acids, proteins, and any combination thereof.

14. The method of claim 10, wherein the analytes within the analyte-containing portions are detected and quantified using at least one assay selected from whole genome sequencing, next generation DNA sequencing, next generation RNA sequencing, PCR, Western blot targeted capture, multiplex PCR, methylation & 16S droplet PCR, and any combination thereof.

15. The method of claim 10, wherein isolating the analyte-containing portions from the surgical drainage fluid sample and the blood sample comprises filtering the samples, centrifuging the samples, contacting the sample with chromatography media, and any combination thereof.

16. The method of claim 10, wherein obtaining the surgical drainage fluid sample from the subject further comprises capturing surgical drainage fluid from a drainage tube associated with the surgery.

17. The method of claim 16, wherein obtaining the surgical drainage fluid sample from the subject further comprises capturing the surgical drainage fluid from the drainage tube within about 24 hours of the surgery.

18. A method for selecting at least one adjuvant treatment for oropharyngeal squamous cell carcinoma (OPSCC) following a resection surgery, the method comprising:
a. obtaining a surgical drainage fluid sample from the surgical site and a blood sample from the subject;
b. isolating analyte-containing portions from the surgical drainage fluid sample and the blood sample;
c. detecting and quantifying HPV viral DNA levels within the analyte-containing portions to produce at least one assay result, wherein the at least one assay result is indicative of oropharyngeal squamous cell carcinoma (OPSCC) within the subject; and
d. selecting the at least one adjuvant treatment based on the at least one at least one assay result.

19. The method of claim 18, wherein selecting the at least one adjuvant treatment based on the at least one at least one assay result further comprises:
a. selecting active surveillance with imaging or no adjuvant treatment if both plasma and SDF HPV levels are below a threshold value; or
b. selecting an adjuvant treatment comprising at least one of radiotherapy, chemotherapy, follow-up surgery, and any combination thereof if at least one of the plasma HPV level and the SDF HPV level are above a threshold value.

20. The method of claim 18, further comprising predicting a time of recurrence or a survival period of the patient based on a predetermined correlation between at least one of the plasma HPV level, the SDF HPV level, and any combination thereof.
